(19) United States
(12) Patent Application Publication
(10) Pub. No.: US 2026/0297524 A1
(43) Pub. Date: Oct. 1, 2026

(54) METHOD FOR MODULATING THE DIFFERENTIATION AND FUNCTIONS OF HUMAN DENDRITIC CELLS

(71) Applicants: INSTITUT NATIONAL DE LA SANTÉ ET DE LA RECHERCHE MÉDICALE, Paris (FR); UNIVERSITÉ D'AIX MARSEILLE, MARSEILLE (FR); CENTRE NATIONAL DE LA RECHERCHE SCIENTIFIQUE, Paris (FR); INSTITUT CURIE, PARIS (FR)

(72) Inventors: Marc DALOD, Marseille (FR); Nicolas MANEL, PARIS CEDEX 05 (FR); Xinlong LUO, Shanghai (CN)

(21) Appl. No.: 19/478,339

(22) PCT Filed: Apr. 26, 2024
(86) PCT No.: PCT/EP2024/061636

§ 371 Date: Oct. 24, 2025

(30) Foreign Application Priority Data
Apr. 26, 2023 (EP) 23315113.3

(51) Int. Cl. C12N 5/0784; A61K 35/15; A61K 40/19; C12N 15/867

(52) U.S. Cl. CPC C12N 5/0639; A61K 35/15; A61K 40/19; C12N 15/867; C12N 2501/125; C12N 2501/145; C12N 2501/727; C12N 2506/11; C12N 2510/00; C12N 2740/15043

(57) ABSTRACT

Type 1 conventional dendritic cells (cDCIs) and plasmacytoid dendritic cells (pDCs) are thought to be critical for anti-tumor or antiviral immunity. In vitro differentiation systems have unlocked the ability to produce large numbers of these cells. However, a method is lacking to systematically identify the cell-intrinsic factors controlling their differentiation and functions that remain therefore poorly understood, in contrast to the situation in mice. Here, the inventors developed a workflow for efficient gene silencing and its tracing in human cDCIs/pDCs generated in vitro. They demonstrate the key role of IRF8 in their development, and of IRF7/MyD88 in human pDC production of interferons-α/λ. They found that SAMHD1 and RAB7B promote human cDC1 differentiation, while SEPT3 promotes human pDC differentiation. Finally, they identified that the transcription factors ID2 and DC-SCRIPT versus BCL11A promote cDC1 versus pDC development respectively. This approach will enable broader genetic screens to advance our understanding of human cDC1s/pDCs and harness them against viral infections or cancer. Thus, the present invention relates to a method for inducing the differentiation of human hematopoietic stem cells into type 1 conventional dendritic cells or plasmacytoid dendritic cells, while genetically manipulating the hematopoietic stem cells to modulate the differentiation and/or functions of their dendritic cell progeny.

### FIELD OF THE INVENTION

The present invention relates to methods for inducing the differentiation and modulating the functions of human type 1 conventional dendritic cells (cDC1s) and human plasmacytoid dendritic cells (pDCs).

### BACKGROUND OF THE INVENTION

The family of dendritic cells (DCs) regroups distinct types of mononuclear phagocytes that have in common a unique efficiency at activating naïve antigen-specific T cells upon their first encounter with their target antigen, a process coined T cell priming¹. DCs encompass plasmacytoid dendritic cells (pDCs) and conventional dendritic cells (cDCs). pDCs are the main producer of type I (α/β) and III (μ) interferons (IFNs) upon the sensing of viral-type stimuli. cDCs are the most efficient cells for T cell priming and are further classified into cDC1s and cDC2s². cDC1s excel in the priming of cytotoxic CD8 T cells, including via their unique efficacy at engulfing and processing antigens from dying cells, to present them in association with the class I major histocompatibility complex (MHC-I) molecules. This process, coined antigen cross-presentation, is crucial for immune defenses against cancer and intracellular pathogens¹. cDC2s rather specialize in the recognition of, and defense against, extracellular parasites (e.g. worms), bacteria and fungi, via the priming of helper CD4 T cells and their functional polarization for the production of type 2 cytokines (helper type 2 or Th2 responses) or of interleukin-17 (Th17)¹.

Most of the mechanistic knowledge we have on the functions of DCs and their molecular regulation has been acquired by using either mouse models³ or human DCs derived in vitro from monocytes (MoDCs)⁴. Human and mouse DCs populations share transcriptional signatures⁵. However, deterministic factors identified in mice do not necessarily play the same role in the human immune system and anti-microbial defense (e.g. TLR3⁶, IL23⁷ or IRF1⁸). MoDCs are a very valuable model that has led to major advances in our understanding of the molecular mechanisms underpinning key DC functions, including antigen processing and presentation, T cell priming and functional polarization. Yet, MoDCs strongly differ from cDCs on many accounts, including their overall molecular make-up^{5, 9}, their adjuvant responsiveness¹⁰, their susceptibility to viral infections¹¹ and their migratory behavior^{12, 13}. Overall, MoDCs appear to be less efficient than cDC1s or even cDC2s or pDCs in promoting CD8 T cell responses against cancer, both in mice¹⁴ and in humans^{15, 16, 17}. Hence, MoDCs are not an adequate surrogate model for many aspects of cDC1 biology.

The low frequency of cDCs and pDCs in all human tissues and their frailness has impeded their study ex vivo as well as their use in the clinic for adoptive cell therapy, in particular for cDC1s. Human DC immunodeficiencies provide a forward-genetic-based approach to deciphering human DC biology¹⁸.

Different methods have been reported to generate in vitro human pDCs, cDC1s or cDC2s¹⁹, including a cost-effective, highly efficient and feeder layer-dependent protocol for in vitro differentiation of human cord blood (CB) CD34⁺ hematopoietic stem cells (HSCs) into bona fide cDC1 and pDCs that the inventors developed recently^{20, 21}. However, likewise to their in vivo counterparts, these in vitro derived cDC1s and pDCs are highly resistant to viral infection or transduction¹¹, making very challenging their genetic manipulation for deciphering the molecular mechanisms regulating their development and functions. There is still a need to improve the differentiation of HSCs in order to generate more human pDCs and cDC1s.

### SUMMARY OF THE INVENTION

The present invention relates to an in vitro or ex vivo method for modulating the differentiation and function of human hematopoietic stem cells (HSCs) into human type 1 conventional dendritic cells (cDC1s) or human plasmacytoid dendritic cells (pDCs), comprising the steps of i) transfecting or transducing a population of human hematopoietic stem cells (HSCs) in vitro or ex vivo with at least one vector encoding at least one gene selected from IRF8, BATF3, SPI1, ID2, DC-SCRIPT, MYD88, TRIF, CD40, IL12B, IL10, PD-L1, PD-L2, TRIM3, RAB7B, RAB5A, SAMHD1, PPT1, IRF7, TCF4, BCL11A, ICOS-L, RAB7 and SEPTIN 3; and ii) culturing the population of HSCs obtained at step i) in at least one dendritic cell induction medium to produce a population of human dendritic cells enriched with type 1 conventional dendritic cells (cDC1s) or plasmacytoid dendritic cells (pDCs) and modulated towards enhanced immunogenicity or on the contrary towards tolerogenic functions.

In particular, the present invention is defined by the claims.

### DETAILED DESCRIPTION OF THE INVENTION

The inventors aimed at adapting their pipeline for cDC1/pDC differentiation by transducing the expanded HSCs prior to inducing their differentiation into cDC1s and pDCs, instead of transducing the already differentiated cDC1s and pDCs. They optimized different parameters to obtain a high transduction rate of both the expanded HSCs and their cDC1/pDC progeny, without any strong perturbation of the expansion and differentiation of the cells, and no spontaneous activation of the obtained cDC1s/pDCs, when using control lentivectors. Importantly, the lentivirus-based approach enables the tracing of the modified cells that is required to ensure the cell-intrinsic nature of gene activities. They validated this pipeline by studying upon gene knock-down in vitro the impact of the IRF8 and IRF7 genes on the development or adjuvant responsiveness of cDC1s/pDCs. They performed a small-scale screen by knocking-down 10 candidate genes and assessing whether it impacted cDC1 or pDC biology, including (1) their development, (2) their ability to produce innate cytokines in response to Toll-like receptor triggering, and (3) their sensitivity to infection by type 1 human immunodeficiency virus (HIV-1). They were able to demonstrate or the first time in human DCs that the three transcription factors ID2, DC-SCRIPT promote human cDC1 development and BCL11 the development of human pDCs.

Their novel pipeline for gene silencing during the in vitro differentiation of HSCs into bona fide human cDC1s and pDCs enabled to (1) extend to humans that the transcription factors ID2 and DC-SCRIPT versus BCL11A promote cDC1 versus pDC development respectively, (2) unravel a novel role of SAMHD1 in balancing HSC differentiation into cDC1s versus pDCs, (3) identify small GTPases that control the development or activation of human cDC1s or pDCs, (4) extend to pDCs the restriction factor activity of RAB15 previously discovered in cDC1s, and demonstrate that RAB7 and BCL11A also limit HIV-1 replication in cDC1s and pDCs.

Accordingly, a first aspect of the invention relates to an in vitro or ex vivo method for modulating the expression of at least one gene of interest in human type 1 conventional dendritic cells (cDC1s) or human plasmacytoid dendritic cells (pDCs) comprising the steps of i) transfecting or transducing a population of human hematopoietic stem cells (HSCs) with at least one gene of interest and/or an inhibitor of the expression of at least one gene of interest and ii) culturing the population of HSCs obtained at step i) in at least one dendritic cell differentiation medium to produce a population of human dendritic cells enriched in type 1 conventional dendritic cells (cDC1s) or plasmacytoid dendritic cells (pDCs).

The method of the invention can be suitable to modulate the differentiation of type 1 conventional dendritic cells (cDC1s) or plasmacytoid dendritic cells (pDCs).

Accordingly, the invention relates to an in vitro or ex vivo method for inducing the differentiation of human type 1 conventional dendritic cells (cDC1s) or human plasmacytoid dendritic cells (pDCs) comprising the steps of i) transfecting or transducing a population of human hematopoietic stem cells (HSCs) in vitro or ex vivo with at least one gene of interest suitable to promote the differentiation of HSCs into cDC1s or pDCS and/or an inhibitor of the expression of at least one gene of interest suitable to prevent the differentiation of HSCs into cDCIs or pDCS and ii) culturing the population of HSCs obtained at step i) in at least one dendritic cell differentiation medium to produce a population of human dendritic cells enriched in type 1 conventional dendritic cells (cDC1s) or plasmacytoid dendritic cells (pDCs).

In some embodiment, at least one gene of interest suitable to promote the differentiation of human hematopoietic stem cells (HSCs) into human cDC1s is selected from the group consisting in IRF8, BATF3, SPI1, ID2, DC-SCRIPT, RAB7B, RAB5A, PPT1 and SAMHD1.

In some embodiment, at least one gene of interest suitable to promote the differentiation of human hematopoietic stem cells (HSCs) into human cDC1s is selected from the group consisting in IRF8, ID2, DC-SCRIPT, RAB7B, RAB5A and SAMHD1.

In some embodiment, 1, 2, 3, 4, 5 or 6 gene(s) of interest suitable to promote the differentiation of human hematopoietic stem cells (HSCs) into human cDC1s is selected from the group consisting in IRF8, ID2, DC-SCRIPT, RAB7B, RAB5A and SAMHD1.

In some embodiment, at least one gene of interest suitable to prevent the differentiation of human hematopoietic stem cells (HSCs) into human cDCIs is selected from the group consisting in RAB7 and/or SEPT3.

In some embodiment, the at least one gene of interest suitable to promote the differentiation of human hematopoietic stem cells (HSCs) into human pDCs is selected from the group consisting in IRF8, TCF4, BCL11A, PPT1 and SEPT3.

In some embodiment, the at least one gene of interest suitable to promote the differentiation of human hematopoietic stem cells (HSCs) into human pDCs is selected from the group consisting in IRF8 and/or BCL11A.

In some embodiment, the at least one gene of interest suitable to prevent the differentiation of human hematopoietic stem cells (HSCs) into human pDCs is selected from the group consisting in ID2 and/or SAMHD1.

In particular embodiment, the inventions refers to an in vitro or ex vivo method for inducing the differentiation of human hematopoietic stem cells (HSCs) into human type 1 conventional dendritic cells (cDC1s) comprising the steps of i) transfecting or transducing a population of human hematopoietic stem cells (HSCs) with at least one gene selected from the group consisting in IRF8, BATF3, SPI1, ID2, DC-SCRIPT, RAB7B, RAB5A, PPT1 and SAMHD1 and/or with an inhibitor of the expression of a gene selected in the group consisting from RAB7 and SEPT3; and ii) culturing the population of HSCs obtained at step i) in at least one dendritic cell induction medium to produce a population of human dendritic cells enriched with type 1 conventional dendritic cells (cDC1s).

In some embodiments, concerning the method for inducing the differentiation of HSC into human cDC1, the population of hematopoietic stem cells (HSCs) is transfected of transduced with at least 1, 2, 3, 4, 5, 6, 7, 8 or 9 genes selected from the group consisting in IRF8, BATF3, SPI1, ID2, DC-SCRIPT, RAB7B, RAB5A, PPT1 and SAMHD1 and/or with an inhibitor of the expression of 1 or 2 gene selected in the group consisting from RAB 7 and SEPT3.

In some embodiments, concerning the method for inducing the differentiation of HSC into cDC1, the population of hematopoietic stem cells (HSCs) is transfected of transduced with at least 1, 2, 3, 4, 5, 6 or 7 genes selected from the group consisting in IRF 8, ID2, DC-SCRIPT, RAB 7B, RAB 5A, PPT 1 and SAMHD 1 and/or with an inhibitor of the expression of 1 or 2 gene selected in the group consisting from RAB7 and SEPT3.

In some embodiments, concerning the method for inducing the differentiation of HSC into cDC1, the population of hematopoietic stem cells (HSCs) is transfected of transduced with 1, 2, 3, 4 or 5 gene selected from the group consisting in IRF8, BATF 3, SPI 1, ID2 and DC-SCRIPT.

In some embodiments, concerning the method for inducing the differentiation of HSC into cDC1, the population of hematopoietic stem cells (HSCs) is transfected of transduced with 1, 2 or 3 gene selected from the group consisting in IRF 8, BATF3, SPI1, ID2 and DC-SCRIPT.

In some embodiments, concerning the method for inducing the differentiation of HSC into cDC1, the population of hematopoietic stem cells (HSCs) is transfected of transduced with IRF8, ID2 and DC-SCRIPT.

In particular embodiment, the inventions refers to an in vitro or ex vivo method for inducing the differentiation of human hematopoietic stem cells (HSCs) into human plasmacytoid dendritic cells (pDCs) comprising the steps of i) transfecting or transducing a population of human hematopoietic stem cells (HSCs) with least one gene selected from IRF8, TCF4, BCL11A, PPT1 and SEPT3, and/or with an inhibitor of the expression of a gene selected in the group consisting from ID2 and SAMHD1; and ii) culturing the population of HSCs obtained at step i) in at least one dendritic cell differentiation medium to produce a population of human dendritic cells enriched with plasmacytoid dendritic cells (pDCs).

In some embodiments, concerning the method for inducing the differentiation of HSC into pDCs, the population of hematopoietic stem cells (HSCs) is transfected of transduced with 1, 2, 3, 4 or 5 gene selected from the group consisting in IRF8, TCF4, BCL11A, PPT1 and SEPT3, and/or with an inhibitor of the expression of 1 or 2 gene selected in the group consisting from ID2 and SAMHD1.

In some embodiments, concerning the method for inducing the differentiation of HSC into pDCs, the population of hematopoietic stem cells (HSCs) is transfected of transduced with IRF8, TCF4, BCL11A, and/or PPT1.

In some embodiments, concerning the method for inducing the differentiation of HSC into pDCs, the population of hematopoietic stem cells (HSCs) is transfected of transduced with IRF8, BCL11A, and/or PPT1.

In some embodiment, the population of human dendritic cells enriched with plasmacytoid dendritic cells (pDCs) exhibit a more efficient immune activity (i. e a higher production of cytokine such as IFN-I) (“more immunogenic pDCs).

In some embodiment, the population of human dendritic cells enriched with type 1 conventional dendritic cells (cDC1s) exhibit a more efficient immune activity (i. e higher production of cytokine and recruitment of T cells) (“more immunogenic cDC1s).

The method of the invention can be suitable to modulate the immune activity of type 1 conventional dendritic cells (cDC1s) or plasmacytoid dendritic cells (pDCs).

Another aspect of the invention relates to an in vitro or ex vivo method for increasing the immune activity of human type 1 conventional dendritic cells (cDC1s) comprising the steps of i) transfecting or transducing a population of human hematopoietic stem cells (HSCs) in vitro or ex vivo with at least one gene of interest suitable to promote the immune activity of cDC1s and/or an inhibitor of the expression of at least one gene of interest suitable to decrease the immune activity of cDC1s and ii) culturing the population of HSCs obtained at step i) in at least one dendritic cell differentiation medium to produce a population of human dendritic cells with more immunogenic type 1 conventional dendritic cells (cDC1s).

The invention relates to an in vitro or ex vivo method for decreasing the immune activity of human type 1 conventional dendritic cells (cDC1s) comprising the steps of i) transfecting or transducing a population of human hematopoietic stem cells (HSCs) in vitro or ex vivo with at least one gene of interest suitable to decrease the immune activity of cDC1s and/or an inhibitor of the expression of at least one gene of interest suitable to promote the immune activity of cDC1s and ii) culturing the population of HSCs obtained at step i) in at least one dendritic cell differentiation medium to produce a population of human dendritic cells with less or no immunogenic type 1 conventional dendritic cells (cDC1s).

In some embodiment, the at least one gene of interest suitable to promote the immune activity of cDC1s is selected from the group consisting in SAMHD1, TRIF, MYD88, DC-SCRIPT, CD40, IL12B and SEPT3.

In some embodiment, the at least one gene of interest suitable to promote the immune activity of cDC1s is selected from the group consisting in SAMHD1, TRIF, MYD88, DC-SCRIPT and SEPT3.

In some embodiment, 1, 2, 3, 4, 5 or 6 gene(s) of interest suitable to promote the immune activity of cDCIs is selected from the group consisting in SAMHD1, TRIF, MYD88, DC-SCRIPT and SEPT3.

In some embodiment, the at least one gene of interest is a gene suitable to decrease the immune activity of cDC1s is selected from the group consisting in BCL11A, RAB7B, RAB7, PD-L1, PD-L2, TIM3 and IL-10.

In some embodiment, the at least one gene of interest is a gene suitable to decrease the immune activity of cDC1s is selected from the group consisting in BCL11A, RAB7B and RAB7.

In some embodiment, 1, 2 or 3 gene(s) of interest is a gene suitable to decrease the immune activity of cDC1s is selected from the group consisting in BCL11A, RAB7B and RAB7.

Another aspect of the invention relates to an in vitro or ex vivo method for increasing the immune activity of human plasmacytoid dendritic cells (pDCs) comprising the steps of i) transfecting or transducing a population of human hematopoietic stem cells (HSCs) in vitro or ex vivo with at least one gene of interest suitable to promote the immune activity of pDCs and/or an inhibitor of the expression of at least one gene of interest suitable to decrease the immune activity of pDC1s and ii) culturing the population of HSCs obtained at step i) in at least one dendritic cell differentiation medium to produce a population of human dendritic cells with more immunogenic plasmacytoid dendritic cells (pDCs).

The Invention relates to an in vitro or ex vivo method for decreasing the immune activity of human plasmacytoid dendritic cells (pDCs) comprising the steps of i) transfecting or transducing a population of human hematopoietic stem cells (HSCs) in vitro or ex vivo with at least one gene of interest suitable to decrease the immune activity of pDCs and/or an inhibitor of the expression of at least one gene of interest suitable to promote the immune activity of DCs and ii) culturing the population of HSCs obtained at step i) in at least one dendritic cell differentiation medium to produce a population of human dendritic cells with less or no immunogenic plasmacytoid dendritic cells (pDCs).

In some embodiment, the at least one gene of interest is a gene suitable to promote the immune activity of pDCs is selected from the group consisting in IRF7, MYD88, BCL11A, RAB7, CD40, IL12B and SEPT3.

In some embodiment, the at least one gene of interest is a gene suitable to promote the immune activity of pDCs is selected from the group consisting in IRF7, MYD88, BCL11A, RAB7 and SEPT3.

In some embodiment, 1, 2, 3, 4 or 5 gene(s) of interest is a gene suitable to promote the immune activity of pDCs is selected from the group consisting in IRF7, MYD88, BCL11A, RAB7 and SEPT3.

In some embodiment, the at least one gene of interest is a gene suitable to decrease the immune activity of pDCs is selected from the group consisting in RAB5A, PD-L1, PD-L2, ICOS-L and IL-10.

In some embodiment, the at least one gene of interest is a gene suitable to decrease the immune activity of pDCs is selected from the group consisting in RAB5A and/or ICOS-L.

As used herein the expression “hematopoietic stem cell (HSC)” has its general meaning in the art and refers to adult multipotent stem cells that give rise to all the blood cell types including for example myeloid lineages (monocytes and macrophages, neutrophils, basophils, eosinophils), erythrocytes, megakaryocytes/platelets, and lymphoid lineages (T-cells, B-cells, NK-cells).

In some embodiment, the hematopoietic stem cells is human cord blood hematopoietic stem cells (CB HSCs).

In some embodiment, the human cord blood hematopoietic stem cells is human cord blood CD34⁺ hematopoietic stem cells (CB CD34⁺ HSCs).

As used herein, the term “dendritic cell” or “DC” refers to a sub-type of antigen presenting cells that are characterized at the morphological level by numerous membrane processes that extend out from the main cell body (similar to dendrites on neurons) and at the biochemical level by cell surface expression of MHC class II molecules and lack of expression of one or more of CD3, CD14, CD19, CD56 and/or CD66b. Subsets of dendritic cells express on their cell surface CD1A, CD1C, CD50, CD54, CD58, CD102, CD80 and/or CD86. Some DCs also express toll-like receptors 2, 3, 4, 7 and/or 9. DCs encompass plasmacytoid dendritic cells (pDCs) and conventional dendritic cells (cDCs). pDCs are the main producer of type I (α/β) and III (λ) interferons (IFNs) upon the sensing of viral-type stimuli. cDCs are the most efficient cells for T cell priming and are further classified into cDC1s and cDC2s².

As used herein the term “a population of human dendritic cells enriched in type 1 conventional dendritic cells (cDC1s)” refers to a population of human dendritic cells with at least 35%, 40%, 45%, 50%, 55%, 60%, 65%, 70%, 75%, 80%, 85%, 90%, 95%, 96%, 97%, 98%, or 99% of type 1 conventional dendritic cells (cDC1 s).

As used herein the term “a population of human dendritic cells enriched in plasmacytoid dendritic cells (pDCs)” refers to a population of human dendritic cells with at least 35%, 40%, 45%, 50%, 55%, 60%, 65%, 70%, 75%, 80%, 85%, 90%, 95%, 96%, 97%, 98%, or 99% of plasmacytoid dendritic cells (pDCs).

As used herein, the term “type 1 conventional dendritic cells” (cDC1s) refers to CD141⁺ CLEC9A⁺ cDCs which excel in the priming of cytotoxic CD8 T cells, including via their unique efficacy at engulfing and processing antigens from dying cells, to present them in association with the class I major histocompatibility complex (MHC-I) molecules. cDC1 recognize intracellular pathogens and initiate type 1 immune responses including ILC1, NK cells, and T helper cell 1 induction. Moreover, cDC1 efficiently cross-present extracellular antigens to CD8⁺ T cells and secrete IL-12, making them important for cytotoxic responses to viral infections and tumors.

As used herein, the term “plasmacytoid dendritic cells” (pDCs) has its general meaning in the art and refers to CD123⁺ CLEC4C⁺ cell and the main producer of type I (α/β) and III (λ) interferons (IFNs) upon the sensing of viral-type stimuli.

As used herein, the term “transfection” or “transfecting” as used herein refers to the process of introducing DNA into a cell, thereby, allowing cellular transformation.

As used herein, the term “transduction” or “transducing” as used herein refers to the process of introducing DNA (e.g., formulated DNA expression vector) into a cell via a viral vector, thereby, allowing cellular transformation.

In particular embodiment, the gene and/or the inhibitor of expression is transfected or transduced into HSC via at least one vector, and especially via a viral vector and in particularly a lentivirus vector.

In particular embodiment, the population of human hematopoietic stem cells (HSCs) is transfected via a protocol as detailed in example.

In particular embodiment, the population of human hematopoietic stem cells (HSCs) is transfected via a protocol comprising the steps i) culturing HSCs in a serum-free medium comprising Fms-related tyrosine kinase 3 ligand (FTL3L), stem cell factor (SCF) and thrombopoietin (TPO) for 24 hours, ii) resuspending the HSCs obtained at step i) in a serum-free medium at a concentration of 1 to 2×10⁵/100 μl, iii) a volume of the HSC suspension obtained in step ii) is seeded with the same volume of a serum-free medium comprising a) Fms-related tyrosine kinase 3 ligand (FTL3L), stem cell factor (SCF) and thrombopoietin (TPO) and b) at least one viral vector comprising at least one gene of interest, wherein the viral vector is at a molarities of infection between 4 and 10.

In particular embodiment, the volume of the HSC suspension seeded in step iii) is a volume comprises between 50 μl and 700 μl, and in particular between 100 μl and 500 μl.

In particular embodiment, the volume of the HSC suspension obtained in step ii) seeded in step iii) is 100 μl.

In particular embodiment, the serum-free medium is X-VIVO 15 serum-free medium. In particular embodiment, the volume of the HSC suspension obtained in step ii) is seeded in 24 or 96-bottom plate in step iii).

As used herein, the term “vector” is any vehicle capable of facilitating the transfer of the nucleic acid to the cells and typically CD34⁺ HSCs. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a lentivirus.

In some embodiments, the vector is a lentivirus vector.

As used herein, the term “lentivirus” refers to enveloped RNA particles measuring approximately 120 nm in size are efficient drug delivery tools and more particularly gene delivery tools. The LV binds to, and enters into target cells through its envelope proteins which confer its pseudotype. Once the LV has entered into the cells, it releases its capsid components and undergoes reverse transcription of the lentiviral RNA before integrating the proviral DNA into the genome of target cells. Non-integrative lentiviral vectors have been generated by modifying the properties of the vector integration machinery and can be used for transient gene expression. Virus-like particles lacking a provirus have also been generated and can be used to deliver proteins or messenger RNA. LV can be used for example, for gene addition, RNA interference, exon skipping or gene editing. All of these approaches can be facilitated by tissue or cell targeting of the LV via its pseudotype.

In some embodiments, the population of HSC is cultured at least one day in a Serum-free Hematopoietic Cell Medium (such as X-Vivo 15) before the transduction or transfection, wherein the Serum-free Hematopoietic Cell Medium comprises FLT3-L, SCF and TPO.

Method for differentiate HSC into dendritic cells are well known in the art as described in Balan S et al. Large-Scale Human Dendritic Cell Differentiation Revealing Notch-Dependent Lineage Bifurcation and Heterogeneity. Cell Rep. 2018 Aug. 14;24(7): 1902-1915.e6; Luo XL et al. The quest for faithful in vitro models of human dendritic cells types. Mol Immunol. 2020 July;123:40-59 and Luo XL et al. In Vitro Generation of Human Cross-Presenting Type 1 Conventional Dendritic Cells (cDC1s) and Plasmacytoid Dendritic Cells (pDCs). Methods Mol Biol. 2023;2618:133-145.

In particular embodiment, the dendritic cell induction medium comprises Fms-related tyrosine kinase 3 ligand (FTL3L), stem cell factor (SCF) and thrombopoietin (TPO) as described in Balan S et al. Large-Scale Human Dendritic Cell Differentiation Revealing Notch-Dependent Lineage Bifurcation and Heterogeneity. Cell Rep. 2018.

In particular embodiment, the dendritic cell induction medium furthermore comprises OP9/OP9-DL 1 feeder layer.

As used herein the term “inhibitor of the expression of a gene” or “inhibitor of gene expression” refers to a natural or synthetic compound that has a biological effect to inhibit the expression of the gene interest (or the gene transcript: RNA). In particular, the inhibitor of the expression of a gene is antisense oligonucleotide, nuclease, siRNA, shRNA or ribozyme nucleic acid sequence.

Inhibitors of gene expression in the present invention may be based on antisense oligonucleotide constructs. Antisense oligonucleotides, including antisense RNA molecules and antisense DNA molecules, would act to directly block the translation the gene of interest mRNA by binding thereto and thus preventing protein translation or increasing mRNA degradation, thus decreasing the level of the gene of interest. For example, antisense oligonucleotides of at least about 15 bases and complementary to unique regions of the mRNA transcript sequence can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression in the present invention. Gene expression can also be reduced by using small double stranded RNA (dsRNA), or a vector or construct causing the production of a small double stranded RNA, such that the gene of interest expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Inhibitors of gene expression according to the present invention may be based nuclease therapy (like Talen or Crispr).

The term “nuclease” or “endonuclease” means synthetic nucleases consisting of a DNA binding site, a linker, and a cleavage module derived from a restriction endonuclease which are used for gene targeting efforts. The synthetic nucleases according to the invention exhibit increased preference and specificity to bipartite or tripartite DNA target sites comprising DNA binding (i.e. TALEN or CRISPR recognition site(s)) and restriction endonuclease target site while cleaving at off-target sites comprising only the restriction endonuclease target site is prevented.

The guide RNA (gRNA) sequences direct the nuclease (i.e. Cas9 protein) to induce a site-specific double strand break (DSB) in the genomic DNA in the target sequence.

Restriction endonucleases (also called restriction enzymes) as referred to herein in accordance with the present invention are capable of recognizing and cleaving a DNA molecule at a specific DNA cleavage site between predefined nucleotides. In contrast, some endonucleases such as for example Fokl comprise a cleavage domain that cleaves the DNA unspecifically at a certain position regardless of the nucleotides present at this position. Therefore, preferably the specific DNA cleavage site and the DNA recognition site of the restriction endonuclease are identical. Moreover, also preferably the cleavage domain of the chimeric nuclease is derived from a restriction endonuclease with reduced DNA binding and/or reduced catalytic activity when compared to the wildtype restriction endonuclease.

According to the knowledge that restriction endonucleases, particularly type II restriction endonucleases, bind as a homodimer to DNA regularly, the chimeric nucleases as referred to herein may be related to homodimerization of two restriction endonuclease subunits. Preferably, in accordance with the present invention the cleavage modules referred to herein have a reduced capability of forming homodimers in the absence of the DNA recognition site, thereby preventing unspecific DNA binding. Therefore, a functional homodimer is only formed upon recruitment of chimeric nucleases monomers to the specific DNA recognition sites. Preferably, the restriction endonuclease from which the cleavage module of the chimeric nuclease is derived is a type IIP restriction endonuclease. The preferably palindromic DNA recognition sites of these restriction endonucleases consist of at least four or up to eight contiguous nucleotides. Preferably, the type 11P restriction endonucleases cleave the DNA within the recognition site which occurs rather frequently in the genome, or immediately adjacent thereto, and have no or a reduced star activity. The type 11P restriction endonucleases as referred to herein are preferably selected from the group consisting of: Pvull, EcoRV, BamH1, Bcnl, BfaSORF1835P, BfiI, Bgll, Bglll, BpuJ1, Bse6341, BsoB1, BspD6I, BstY1, Cfr101, Ecl18kl, Eco0109l, EcoR1, EcoR11, EcoRV, EcoR124l, EcoR124ll, HinP11, Hincll, Hindlll, Hpy99l, Hpy1881, Mspl, Munl, Mval, Nael, NgoMIV, Notl, OkrAl, Pabl, Pacl, PspGl, Sau3Al, Sdal, Sfil, SgrAl, Thal, VvuYORF266P, Ddel, Eco57l, Haelll, Hhall, Hindll, and Ndel.

Ribozymes can also function as inhibitors of gene expression according to the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of gene expression can be prepared by known methods. These include techniques for chemical synthesis such as, e.g., by solid phase phosphoramadite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

As used herein, the term “IRF8″ or ”Interferon regulatory factor 8″ has its general meaning in the art and refers to a transcription factor playing a critical role in the regulation of lineage commitment and in myeloid cell maturation including the decision for a common myeloid progenitor (CMP) to differentiate into a monocyte precursor cell. Its Entrez number reference is 3394.

As used herein, the term “ID2” or “DNA-binding protein inhibitor ID-2” has its general meaning in the art and refers to an inhibitor of DNA binding (ID) family, which are transcriptional regulators that contain a helix-loop-helix (HLH) domain but not a basic domain. Its Entrez number reference is 3398.

As used herein, the term “DC-SCRIPT” or “Dendritic cell-specific transcript”, also known as Zinc finger protein 366, has its general meaning in the art and refers to a protein that in humans is encoded by the ZNF366 gene, and that was found to be specifically expressed in dendritic cells. Its Entrez number reference is 167465.

As used herein, the term “RAB7B” or “Ras-related protein Rab-7b” has its general meaning in the art and refers to a small GTPase that plays a role in the transport and degradation of proteins in endosomes and lysosomes in mammalian cells. Its Entrez number reference is 338382.

As used herein, the term “RAB5A” or “Ras-related protein Rab-5A” has its general meaning in the art and refers to a small GTPase that plays a role in the maturation of endosomes. Its Entrez number reference is 5868.

As used herein, the term “PPT1” or “Palmitoyl-protein thioesterase 1” has its general meaning in the art and refers to a member of the palmitoyl protein thioesterase family. PPT-1 is a small glycoprotein involved in the catabolism of lipid-modified proteins during lysosomal degradation. Its Entrez number reference is 5538.

As used herein, the term “SAMHD1” or “SAM domain and HD domain-containing protein 1” has its general meaning in the art and refers to a cellular enzyme, responsible for blocking replication of HIV in dendritic cells, [5] macrophages, [6] monocytes[7] and resting CD4⁺ T lymphocytes. Its Entrez number reference is 25939.

As used herein, the term “RAB7” or “Ras-related protein Rab-7a” has its general meaning in the art and refers to a member f the RAB family of RAS-related GTP-binding proteins involved in endocytosis. RAB7 has been localized to late endosomes and shown to be important in the late endocytic pathway. In addition, it has been shown to have a fundamental role in the cellular vacuolation induced by the cytotoxin VacA of Helicobacter pylori. Its Entrez number reference is 7879.

As used herein, the term “SEPT3” or “Neuronal-specific septin-3” has its general meaning in the art and refers to the septin family of GTPases. Members of this family are required for cytokinesis. Its Entrez number reference is 55964.

As used herein, the term “BCL 11A” or “B-cell lymphoma/leukemia 11A” has its general meaning in the art and refers to a regulatory C2H2 type zinc-finger protein, that can bind to the DNA. Its Entrez number reference is 53335.

As used herein, the term “BATF3” or “Basic Leucine Zipper ATF-Like Transcription Factor 3” has its general meaning in the art and refers to a transcription factor that controls the differentiation of CD8⁺ thymic conventional dendritic cells in the immune system. Its Entrez number reference is 55509. As used herein, the term “SPI 1” or “Transcription factor PU.1” has its general meaning in the art and refers to an ETS-domain transcription factor that activates gene expression during myeloid and B-lymphoid cell development. Its Entrez number reference is 6688.

As used herein, the term “TCF4” or “Transcription factor 4” has its general meaning in the art and refers to a transcription factor that primarily involved in neurological development of the fetus during pregnancy by initiating neural differentiation by binding to DNA. Its Entrez number reference is 6925.

As used herein, the term “CD40” or “Cluster of differentiation 40” has its general meaning in the art and refers to a type I transmembrane protein found on antigen-presenting cells and is required for their activation. Its Entrez number reference is 958.

As used herein, the term “TRIM3” or “Tripartite motif-containing protein 3” has its general meaning in the art and refers to a member of the tripartite motif (TRIM) family, also called the ‘RING-B-box-coiled-coil’ (RBCC) subgroup of RING finger proteins. Its Entrez number reference is 10612.

As used herein, the term “IL-12B” or “Subunit beta of interleukin 12” has its general meaning in the art and refers to a subunit of interleukin 12, a cytokine that acts on T and natural killer cells, and has a broad array of biological activities. Its Entrez number reference is 3593.

As used herein, the term “IL-10” or “Interleukin 10” has its general meaning in the art and refers to a cytokine with multiple, pleiotropic, effects in immunoregulation and inflammation. It downregulates the expression of Th1 cytokines, MHC class II antigens, and co-stimulatory molecules on macrophages. Its Entrez number reference is 3586.

As used herein, the term “MyD88” or “Myeloid differentiation primary response 88” has its general meaning in the art and refers to a protein involved in signaling within immune cells. The MyD88 protein acts as an adapter, connecting proteins that receive signals from outside the cell to the proteins that relay signals inside the cell. Its Entrez number reference is 4615.

As used herein, the term “TRIF” or “TIR-domain-containing adapter-inducing interferon-β” has its general meaning in the art and refers to an adapter in responding to activation of toll-like receptors (TLRs). It mediates the rather delayed cascade of two TLR-associated signaling cascades, where the other one is dependent upon a MyD88 adapter. Its Entrez number reference is 106759.

As used herein, the term “PD-L1” or “Programmed death-ligand 1” has its general meaning in the art and refers to a 40 kDa type 1 transmembrane protein that plays a critical role in induction and maintenance of immune tolerance to self. Its Entrez number reference is 29126.

As used herein, the term “PD-L2” or “Programmed cell death 1-ligand 2” has its general meaning in the art and refers to a cell surface receptor belonging to the B7 protein family. It consists of both an immunoglobulin-like variable domain and an immunoglobulin-like constant domain in the extracellular region, a transmembrane domain, and a cytoplasmic domain. Its Entrez number reference is 80380.

As used herein, the term “ICOS-L” or “ICOS ligand” has its general meaning in the art and refers to a glycosylated transmembrane structure, which is classified as a member of the B7 family. The interaction of ICOS-L with ICOS is critically involved in the activation, proliferation, differentiation and cytokine production of T cells as well as in the antibody secretion from B cells during secondary immune responses. Its Entrez number reference is 23308.

In another aspect, the invention refers to a population of human dendritic cells enriched of type 1 conventional dendritic cells (cDC1s) produced by the method of the invention, as described above.

The invention also refers to a population of human dendritic cells enriched of plasmacytoid dendritic cells (pDCs) produced by the method of the invention, as described above.

The invention also refers to a population of human dendritic cells with more immunogenic plasmacytoid dendritic cells (pDCs) produced by the method of the invention, as described above.

The invention also refers to a population of human dendritic cells with more immunogenic cDCIs produced by the method of the invention, as described above.

The invention also refers to a population of human dendritic cells with less immunogenic plasmacytoid dendritic cells (pDCs) produced by the method of the invention, as described above.

The invention also refers to a population of human dendritic cells with less immunogenic cDCIs produced by the method of the invention, as described above.

In another aspect, the invention refers to the population of human dendritic cells enriched of type 1 conventional dendritic cells (cDC1s) of the invention, as described above, for use in therapy.

The invention also refers to the population of human dendritic cells enriched of plasmacytoid dendritic cells (pDCs) of the invention, as described above, for use in therapy.

The invention also refers to a population of human dendritic cells with more immunogenic plasmacytoid dendritic cells (pDCs) produced by the method of the invention, as described above, for use in therapy.

The invention also refers to a population of human dendritic cells with more immunogenic cDCIs produced by the method of the invention, as described above, for use in therapy.

The invention also refers to a population of human dendritic cells with less immunogenic plasmacytoid dendritic cells (pDCs) produced by the method of the invention, as described above, for use in therapy.

The invention also refers to a population of human dendritic cells with less immunogenic cDCIs produced by the method of the invention, as described above, for use in therapy.

In some embodiments, the therapy is adoptive cell therapy in a subject in need thereof. The term “adoptive cell therapy” as used herein refers to a cell-based immunotherapy that relates to the transfusion of autologous or allogenic lymphocytes, genetically modified or not.

The populations of human dendritic cells of the present invention, can be utilized in methods and compositions for adoptive cell therapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the age and weight of the recipient, on the severity of the targeted condition. The number of cells will depend upon the ultimate use for which the composition is intended, as will the type of cells included therein. The clinically relevant number of immune cells can be apportioned into multiple infusions that cumulatively equal or exceed the desired total amount of cells.

As used herein, the term “subject” denotes a mammal, such as a rodent, a feline, a canine, and a primate. Preferably, a subject according to the invention is a human. In some embodiments, the subject is a human having or susceptible to have infectious disease or cancer.

In some embodiment, the hematopoietic stem cells may be isolated from the subject (“autologous cells”) or from another individual (“allogeneic cells”).

As used herein, “allogeneic cells” refers to cells isolated from one subject (the donor) and infused in another (the recipient or host).

As used herein, “autologous cells” refers to cells that are isolated and infused back into the same subject (recipient or host).

In some embodiments, the therapy is cancer therapy or infectious disease therapy.

Thus in other words, the inventions refers to a method of treating cancer in a subject in need thereof, said method comprising administering to the subject a therapeutically effective amount of the population of human dendritic cells enriched of type 1 conventional dendritic cells (cDC1s) of the invention and/or the population of human dendritic cells enriched inplasmacytoid dendritic cells (pDCS) of the invention and/or the population of human dendritic cells with more immunogenic plasmacytoid dendritic cells (pDCs) of the invention and/or with more of the invention and/or the population of human dendritic cells with more immunogenic type 1 conventional dendritic cells (cDC1s) of the invention.

As used herein, the term “therapeutically effective amount” refers to an amount sufficient to produce the desired physiological effect or amount capable of achieving the desired result, particularly for treating the disorder or disease condition, including reducing or eliminating one or more symptoms or manifestations of the disorder or disease. The effective amount will vary with the age, general condition of the subject, the severity of the condition being treated, the nature of any concurrent treatment, the pharmaceutically acceptable carrier used, and like factors within the knowledge and expertise of those skilled in the art. As appropriate, an “effective amount” in any individual case can be determined by one of skill in the art by reference to the pertinent texts and literature and/or by using routine experimentation.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse.

The treatment may be administered to a subject having a medical disorder or who ultimately may acquire the disorder, in order to prevent, cure, delay the onset of, reduce the severity of, or ameliorate one or more symptoms (i. e dry eye) of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “cancer” refers to a condition in which abnormally replicating cells of host origin are present in a detectable amount in a subject. The cancer can be a malignant or non-malignant cancer. Cancers include, but are not limited to, biliary tract cancer; brain cancer; breast cancer; cervical cancer; choriocarcinoma; colon cancer; endometrial cancer; esophageal cancer; gastric cancer; intraepithelial neoplasms; leukemias; lymphomas; liver cancer; lung cancer; melanoma; neuroblastomas; oral cancer; ovarian cancer; pancreatic cancer; prostate cancer; rectal cancer; renal cancer; sarcomas; skin cancer; testicular cancer; thyroid cancer; as well as other carcinomas and sarcomas. Cancers can be primary or metastatic.

In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In another aspects, the inventions refers to a method of treating infectious disease in a subject in need thereof, said method comprising administering to the subject a therapeutically effective amount of the population of human dendritic cells enriched of type 1 conventional dendritic cells (cDC1s) of the invention and/or the population of human dendritic cells enriched of plasmacytoid dendritic cells (pDCs) of the invention and/or the population of human dendritic cells with more immunogenic plasmacytoid dendritic cells (pDCs) of the invention and/or with more of the invention and/or the population of human dendritic cells with more immunogenic type 1 conventional dendritic cells (cDC1s) of the invention.

The invention also refers to a method of treating infectious disease in a subject in need thereof, said method comprising administering to the subject a therapeutically effective amount of the human dendritic cells enriched of plasmacytoid dendritic cells (pDCs) of the invention. As used herein, the term “infectious disease” refers to a condition in which an infectious organism or agent is present in a detectable amount in the blood or in a normally sterile tissue or normally sterile compartment of a subject.

In some embodiments, the infectious disease is caused by an intracellular pathogen.

As used herein the term “intracellular pathogen” has its general meaning in the art and refers to viruses, bacteria, fungi, and parasites capable of growing inside macrophages or other cells of the host and avoiding destruction. Intracellular pathogen includes but are not limited to Chlamydia spp, Rickettsia spp, Mycobacterium leprae, influenza virus (e.g., Influenza virus A, Influenza virus B), respiratory syncytial virus, adenovirus, metapneumovirus, cytomegalovirus, parainfluenza virus (e.g., hPIV-1, hPIV-2, hPIV-3, hPIV-4), rhinovirus, coxsackie virus, echo virus, herpes simplex virus, coronavirus (SARS-coronavirus such as SARS-Cov1 or SARS-Cov2), human Immunodeficiency Viruses (HIV-1, HIV2), smallpox, Plasmodium spp, Toxoplasma gondii, Babesia spp, Leishmania donovani, and Trypanosoma cruzi

In some embodiments, the infectious disease is a viral infection.

The term “viral infection” therefore also includes any clinical sign, symptom or disease that occurs in an animal or human (patient) following contamination of said animal or patient by a virus as described in the present application. Accordingly, the “viral infection” includes both contamination by said virus and the various pathologies which are the consequence of contamination by said virus.

In particular embodiment, the viral infection is caused by a virus selected from the group consisting of influenza virus (e.g., Influenza virus A, Influenza virus B), respiratory syncytial virus, adenovirus, metapneumovirus, cytomegalovirus, parainfluenza virus (e.g., hPIV-1, hPIV-2, hPIV-3, hPIV-4), rhinovirus, coxsackie virus, echo virus, herpes simplex virus, coronavirus (SARS-coronavirus such as SARS-Cov1 or SARS-Cov2), human Immunodeficiency Viruses (HIV-1, HIV2) and smallpox.

In some embodiments, the population of human dendritic cells with less immunogenic plasmacytoid dendritic cells (pDCs) and/or the population of human dendritic cells with less immunogenic cDC1s are suitable to treat auto-immune disease.

Thus in other words, the inventions refers to a method of treating cancer in a subject in need thereof, said method comprising administering to the subject a therapeutically effective amount of the population of human dendritic cells enriched of type 1 conventional dendritic cells (cDC1s) of the invention and/or the population of human dendritic cells enriched inplasmacytoid dendritic cells (pDCS) of the invention and/or the population of human dendritic cells with more immunogenic plasmacytoid dendritic cells (pDCs) of the invention and/or with more of the invention and/or the population of human dendritic cells with more immunogenic type 1 conventional dendritic cells (cDC1s) of the invention.

As used herein the term “autoimmune disease” has its general meaning in the art and refers to an abnormal immune response against functioning body part, i. e against a substance that does not normally elicit an immune response in a healthy subject. The causes of autoimmune disorders are not well understood and many have no cure. Examples of autoimmune diseases include e.g. rheumatoid arthritis (RA), systemic lupus erythematosus (lupus), inflammatory bowel disease (IBD), multiple sclerosis (MS), type 1 diabetes mellitus, Guillain-Barre syndrome, Crohn's disease and psoriasis. Many of these diseases are chronic and can cause significant morbidity and disability. Treatment of autoimmune diseases is generally based on immunosuppression.

The invention will be further illustrated by the following figures and examples. However, these examples and figures should not be interpreted in any way as limiting the scope of the present invention.

### FIGURES

FIG. 1: Impact of candidate transcription factors on the development and activation of human pDCs and cDC1s. Impact of the knock-down of ID2 (A), DC-SCRIPT (B) and BCL11A (C), on human cDC1 and pDC development. (A, B, C) for ID2, the results are shown from seven independent experiments, with five distinct CB donors and two different shRNA (full versus empty symbols); for BCL11A: ten independent experiments, with six CB donors and one shRNA; for DC-SCRIPT: four independent experiments, with four CB donors and one shRNA. Statistical analyses were performed using a two-tailed non-parametric Mann-Whitney test. *, p<0.05; **, p<0.01; ***, p<0.001; **** p<0.0001; ns, not significant.

FIG. 2: Identification of small GTPases controlling the development or activation of human cDC1s or pDCs. A-D) Impact on the development of human cDC1s or pDCs of the knock-down of RAB7B (A), RAB5A (B), RAB7 (C) and SEPT3 (D). For the impact of RAB7B on DC development, seven independent experiments are shown, with four CB donors, and one shRNA; for RAB5A, three independent experiments, with two CB donors, and two different shRNA; for RAB7, ten independent experiments, with seven CB donors, and two different shRNA; for SEPT3, eight independent experiments, with six CB donors, and three different shRNA. Statistical analyses were performed using a two-tailed non-parametric Mann-Whitney test. *, p<0.05; **, p<0.01; ***, p<0.001; ****, p<0.0001; ns, not significant.

FIG. 3: Identification of antiviral restriction factors controlling the development of human cDC1s or pDCs. Impact on cDC1 or pDC development of the knock-down of SAMHD1 (A), RAB15 (B) and PPT1 (C). The results are shown from seven independent experiments, with five CB donors and one shRNA for RAB15; from eight independent experiments, with six CB donors and three different shRNA for SAMHD1; from six independent experiments, with four CB donors and three different shRNA for PPT1. Statistical analyses were performed using a two-tailed non-parametric Mann-Whitney test. *, p<0.05; **, p<0.01; *, p<0.001; *, p<0.0001; ns, not significant.

### EXAMPLE

### Material & Methods

### Cells and Antibodies

Human CB CD34+ HSCs were bought from ABCell-bio, France. For each purchasing campaign, a tube of 200,000 cells was first ordered for a series of donors, to test their differentiation efficacy into cDC1s and pDCs, before ordering additional tubes of between 300,000 and 700,000 cells for the donors yielding the most efficient differentiation.

### Lentivector Production and Concentration

pLKO.1-GFP plasmid was adapted to pLKO.1-mTagBFP by replacing the CDS of GFP with that of mTagBFP. Briefly, CDS of mTagBFP was cloned from pTRIF-SFFV-mTagBFP-2A plasmid79. The amplified fragment and pLKO.1-GFP plasmid were digested with NsiI and BamHI at 37° C. overnight. Digested fragment and plasmid were then ligated and used to transform competent E. coli cells. Positive clones were selected and verified by using bacterial-liquid PCR and sequencing. mTagBFP expression measured by flow cytometry in 293 FT cells transfected with the plasmid. The packaging ability of the lentivectors was also verified to ensure that it did not differ from that of the parental pLKO.1-GFP vectors. 293 FT cells were seeded into 6-well plates. The transfection was performed by combining 0.3 μg pVSV-G, 0.9 μg pPAX2 (psPAX2 was a gift from Didier Trono (Addgene plasmid #12260 ; http://n2t.net/addgene:12260 ; RRID:Addgene_12260) and 1.05 μg LKO.1 -shRNA-BFP as previously described11. The next day, the supernatant was discarded and replaced by 3 ml of X-VIVO 15 serum-free medium. Two days after, the supernatant was harvested into 50 ml Falcon tubes. A small aliquot of the virus (500 μl) was used for titration. The bulk of the supernatant was stored at μ80° C. until the lentivector titration was known, then it was thawed for concentration via ultracentrifugation after the lentivectors were titrated. To construct the plasmid for IRF8 overexpression, CDS of IRF8 from IRF8 human tagged ORF clone (RG217646, Origene) were cloned and digested with BamHD1 and XhoI. The fragment was then cloned into TRIP-SFFV-mTagBFP-2A (Addgene, #102585). To produce lentivectors for IRF8 overexpression, transfection of previously seeded 293 FT cells was performed by combining 0.3 μg pVSV-G, 0.9 μg psPAX2 and 1.05 μg pTRIF-SFFV-IRF 8 -mTagBFP or empty vector for negative control. For titration, briefly, serial dilutions of the lentivectors were made and added to previously prepared 293 FT cells plated in 96 well flat bottom plates at 5,000 cells/well. Forty-eight hours after, cells were harvested by Trypsin digestion and resuspended into PBS. The infection rate in each well were measured by FACS. The titers of the lentivectors were calculated as previously reported80, 81, as infectious units (IU) per mL, based on the percentages of BFP+ or GFP+ cells and on Poisson distribution (—In(100-% BFP+ or GFP+)/100); only values ranging between 1% and 40% were used for calculation to ensure that they felled into a linear distribution. For concentrating them, the lentivectors were thawed and ultracentrifugated at 19,000 rpm for 2 hours at 4° C. The supernatant was then discarded and the pellets resuspended with the volume calculated to achieve a final lentivector titer of 107 IU/ml. The resuspended concentrated lentivectors were then aliquoted at 100 μl/tube and stored at −80° C. until use.

### Transduction of THP-1 Cells

Cultured THP-1 cells were harvested and resuspended into 106 per ml. 500 μl of the cells were seeded into 24 well plate and 500 μl of nonconcentrated lentivectors were added, complemented with polybrene, the transduction was performed via spinoculation at 800 g, 2 hours under room temperature. After spinoculation, cells were put back into the inbubator and cultured for one week. The cells were finally cultured in 90 mm petri dish before harvest due to the quick expansion.

RNA Extraction and qRT-PCR

Before RNA extraction, the transduction rate were measured by flow cytometry to ensure that enough percentage of cells are transduced (>60%). The cells were then pelleted into a RNase-free Eppendorf tubes and were lysed in 1 ml Trizol reagent (Invitrogen, Ref. 15596026). 10 minutes after the addition of Trizol, 200 μl chloroform were then added. The mixtures were then thoroughly mixed and keep them in room temperature before centrifugation at 12000 rpm at 4° C. for 15 mins. The upper clear fractions were then transferred to new RNase free eppendorf tubes (around 450 μl) and 500 μl isopropanol were then added and thoroughly mixed. The tubes were kept at room temperature for another 10 minutes before centrifugation at 12000 rmp under 4° C. for 10 mins. After the centrifugation, the supernatant was discarded and the pellets were washed with 1 ml 70% ethanol by centrifugating at 12000 rmp at 4° C. for 5 mins. The supernatant was then discard and the pellets were then dried under an RNase-free chemical hood for 20 mins and 100 μl RNase-free water were not added until the residual liquid could not be observed. After complete dissolve, the concentration was measured by NanoDrop 2000/2000c Spectrophotometer (Thermo fisher) and the concentrations of RNA were finally adjusted to 100 ng/μl. cDNA was prepared from 2 μg total RNA from each sample under the instruction of Quanti Tech reverse transcription Kit (Qiagen, 205311). Samples for QPCR were then prepared under the instruction of ONEGreen fast qPCR kit (Ozyme, OZYA008-40) and loaded on ABI 7500 fast Real-time PCR system. The relative mRNA of each gene was normalized to the mRNA level of GAPDH in each sample.

### Western Blot

For analysis the knocking down efficiency of the shRNA candidates for different genes including SAMHD1, RAB7B, RAB7, RAB5A, PPT1, BCL11A, IRF8 and IRF5, shRNA transduced THP-1 cell lysates were prepared, while for SEPT3, shRNA transduced HEK293FT cell lysates were prepared. 5×10⁶ cells were pelleted after detection of the transduction rate by flow cytometry and were washed in PBS and lysed in 200 μl of Pierce RIPA buffer (Thermo Scientific, 89900), complemented with protease inhibitor (Roche; 1187358001)) on ice for 30 mins. 40 μl of 6X Laemmli buffer (4% SDS, 20% Glycerol, 0.125 M Tris-HCl pH6.8 , 10% 2-mercaptoehtanol, 1% bromophenol blue) was added and samples were boiled at 95° C. for 20 min. 40 μl of cell lysates were loaded on previously-prepared 10% SDS-PAGE gels and transferred on nitrocellulose membrane after the membrane was saturated 30 min at room temperature with transferring buffer. The membranes were then blocked with 0.5% (w/v) non-fat milk powder in TBS buffer. Proteins were blotted with antibodies against SAMHD1 (1:1000), RAB7B (1:1000), RAB7 (1:1000), RAB5A (1:1000), PPT1 (1:1000), BCL11A (1:1000), IRF8 (1:1000) and IRF5 (1:1000) and actin (1:10000) in 0.25% (w/v) non-fat milk powder in TBS overnight at 4° C. in TBS buffer. Membranes were washed three times 10 min in TBS with 0.1% Tween (TBS-T) and incubated for 1 hr with secondary goat antibodies against mouse (1/10000) or donkey antibodies against rabbit (1/5000) in 0.25% (w/v) non-fat milk powder in TBS buffer. ECL signal was obtained with Pierce western blot substrate (Thermo Scientific, 32106) and was recorded on the ChemiDoc XRS Imager (Biorad).

### Transduction of Expanded HSCs

The HSCs were expanded as reported previously²⁰. The transduction protocol was divided into three steps. 1) The first step was the “priming phase”. One vial of expanded HSCs (100,000-400,000 cells) was thawed and suspended into 1 ml X-VIVO 15 serum free medium and put into one well of a 24-well plate. Cytokines (FST, meaning FLT3-L, SCF and TPO) were added at the final concentration of 100 ng/ml. Cells were then incubated at 37° C., 5% CO2 for 24 hours. 2) The second step was the transduction itself. The pre-stimulated cells were harvested and resuspended in X-VIVO 15 serum-free medium at the concentration of 1 to 2×10⁵/100 μl. 100 μl of the cell suspension was seeded per well of a 96-well U-bottom plate. Then, each well received 100 μl of concentrated lentivectors (~10⁷ IU/ml in X-VIVO 15 serum free medium supplemented with FST to reach the same final concentration as for the priming phase). Finally, poxamer 407 (Sigma) was added to each well, at a final concentration of 100 μg/ml. 3) The third step consisted in seeding the cells to induced their differentiation into cDC1s/pDCs. 24 hours after step 2, the transduced cells were harvested and counted. 2×10⁴ cells were seeded onto the previously prepared OP9/OP9-DL1 feeder layer, in 24 well plates, as previously reported²⁰. Half of the supernatant was changed once a week with fresh medium containing FST, as previously described²⁰. Two to three weeks after initiation of the differentiation phase, the cells were harvested for analysis of the proportion sof cDC1s/pDCs or for performing TLR stimulation or HIV-1 infection experiments.

### Definition of “Differentiation Efficacy”

To define the “differentiation efficacy” of cDC1s and pDCs, we first calculated the ratio of the percentages of total BFP⁺ cells to the total BFP cells as “ratio total” in all live and CD45⁺ cells. The ratio of the percentages of BFP⁺ pDCs to BFP⁻ pDCs as “ratio_pDCs” and that of cDC1s as “ratio_cDC1s”. The pDC differentiation efficacy was then calculated by dividing ratio_pDCs with ratio_total, and cDC1 differentiation efficacy was calculated by dividing ratio cDC1 with ratio_total. In most of the experiments, values of differentiation efficacy from shLacZ-transduced samples were around 1 as expected for lack of specific effect of transduction in pDCs or cDCIs as compared to the other CD45⁺ cells in the cultures. However, due to the donor differences or fluctuations in the culture system, values of differentiation efficacy from shLacZ-transduced samples were sometimes as low as 0.5 or as high as 1.5. In these cases, these values were normalized to 1 and the values for the test shRNA-transduced samples in the same experiments were normalized consistently.

### Stimulation With Synthetic TLR Ligands

To activate DCs, 5 μg/ml R848 or 10 μg/ml polyI:C were added to the culture, followed one hour later by the addition of BFA to block exocytosis and hence enable intracellular detection of cytokines by flow cytometry. Five hours after the addition of the TLR ligands, cells were harvested and stained for FACS detection of TNF, IFN-α or IFN-λ in cDC1s and pDCs. The effect of gene knock-down on cDC1 or pDC activation was assessed by calculating the ratio of the percentage of cytokine-producing cells within BFP⁺ cDC1s/pDCs to the percentage of cytokine-producing cells within BFP-cDC1s/pDCs.

### HIV-1 Production and Infection

All species of HIV-1 were produced by transfecting HEK293FT cells that were seeded in 6-well plates in BSL-3, using the TransIT-293 Transfection Reagent (Mirus Bio, USA) and following the instruction of the manufacturer. For the production of HIV-1R5GFP, 2.25 μg pR5GFP were used for each well. For HIV-1 R5GFP^{Vpx}, 0.3 μg pIRES2EGFP-VPXany Vpr+ 1.75 μg pR5GFP were mixed for each well. To mutate the wild-type envelop (BAL) on pR5GFP, overlap PCR was used to introduce TA right after the N-terminal 249 bases in order to create a stop codon with the sequential G nucleoside and hence terminate the translation process. The pR5GFP with deficient envelop gene were named pR5GFP^{ΔBAL} and verified for their inability to produce infectious viral particles. For the production of VSV-G pseudo typed R5GFP^{ΔBAL}, 0.3 μg pCMV-VSV-G+ 1.95 μg pR5GFP^{ΔBAL} were mixed for each well. For VSV-G pseudo typed R5GFP^{ΔBAL+Vpx}, 0.3 μg pCMV-VSV-G+0.3 μg pIRES2EGFP-VPXany Vpr+1.65 μg pR5GFP^{ΔBAL} were mixed for each well. The next day after the transfection, the supernatant were replaced with 3 ml pre-warmed differentiation medium (Δ-MEM Glutamax, 10% FCS, 1% penicillin-streptomycin, 1X NEAA, 2 mM L-Glutamin, 10 mM HEPES, 1 mM sodium pyruvate and 50 μM β-mercaptoethanol). The virus containing supernatant were harvested at two days post-transfection, filtered at 45 μm, aliquoted into 1 ml Eppendorf tubes and stored at −80° C. For the infection of in vitro differentiated DC cultures in 24 well plates, 500 μl of supernatant were removed and 320 μl fresh medium supplemented with cytokine cocktails for the differentiation were added. 180 μl of virus were then added, with protamine supplementation at a final concentration of 1 μg/ml. Infection was performed under spinoculation at 800 g for two hours. Plates were put back into the incubator for 48 hours before staining for assessing by flow cytometry the infection rate of the different cell types.

### Blockade of the IFN-I Response

Vaccinia virus B18R carrier-free recombinant protein (eBioscience, 34-8185-81) was purchased and used at 3 μg/ml as a type I IFN blocker, as previously described¹¹.

### Bulk-RNA Sequencing

The differentiated cDC1s and pDCs from two CB donors were sorted by flow cytometry to a purity>98%. The concentration and integrity of each RNA sample was verified by using Agilent RNA 6000 Nano chips and analyzing them with the 2100 bioanalyzer according to the manufacturer instructions. Bulk RNA sequencing was then performed by the sequencing core facility of the Curie Institute in Paris and the results analyzed as previously described¹¹.

### Statistical Analysis

No statistical methods were used to pre-determine sample sizes but our sample sizes are similar to those reported in previous publications^{11, 31}. Data distribution was assumed to be normal but this was not formally tested. All quantifications were performed with awareness of experimental conditions, meaning not in a blinded fashion. No data were excluded. Statistical parameters including the definitions and exact value of n (number of biological replicates and total number of experiments), and the types of the statistical tests are reported in the figures and corresponding legends. Statistical analyses were performed using Prism v8.1.2 (GraphPad Software). Statistical analysis was conducted on data with at least three biological replicates. Comparisons between groups were planned before statistical testing and target effect sizes were not predetermined. Error bars displayed on graphs represent the mean±SEM. Statistical significance was defined as * for P<0.05, ** for P<0.01, *** for P<0.001 and **** for P<0.0001.

### Results

Pipeline Development to Genetically Edit Human cDC1s/pDCs

We confirmed that differentiated cDC1s and pDCs were largely refractory to lentiviral transduction, even when using concentrated lentivectors (data not shown). To overcome this technical bottleneck, we optimized our protocol to achieve a high transduction rate of CB HSCs, with maintenance of a high proportion of transduced cells in their differentiated pDC and cDC1 progeny (data not shown). We first tested modifications of the medium and cytokines used for the culture of the HSCs, to select conditions promoting a higher transduction efficacy. Using X-VIVO 15 serum-free medium without supplements, and the cytokine/growth factor cocktail FLT3L/SCF/TPO (FST), provided a better transduction rate than the four other culture conditions tested (data not shown). The transduction rate of the HSCs was further increased by optimizing five additional parameters (data not shown): (i) using higher molarities of infection (MOI) upon increasing lentivector titers by concentration, (ii) promoting cell-to-cell contacts upon using 96-well U-bottom plates instead of 24-well plates, and hence also (iii) performing the transduction in a small volume, (iv) replacing protamine with poloxamer 407 that had been reported to promote higher transduction rates of HSCs⁶², and (v) adding to our culture protocol a “priming phase” by culturing expanded HSCs in the transduction medium without poloxamer (“priming medium”) for one day prior to their exposure to lentivectors. This optimized protocol yielded higher percentages of transduced HSCs (up to 90%) and also promoted a better cell survival, as compared to the semi-optimized protocol (data not shown). After two to three weeks of differentiation, the percentages of the transduced cells were maintained at high levels, similar to those of their parental HSCs that had been transduced with the optimized protocol (data not shown). Seeding more HSCs on the feeder layer increased their production of cDC1s during the differentiation (data not shown). Hence, we selected to seed 2×10⁴ HSCs/ml for the differentiation phase, as the best compromise between the cost of the HSCs and the increase in their differentiation into cDC1s. Under these experimental conditions, when using BFP-expressing shRNA lentivectors, both transduced and non-transduced cDC1s/pDCs were clearly detectable by flow cytometry in the same culture well (data not shown). Thus, it allowed comparing the gene-modified versus control cells side-by-side, to assess the cell-intrinsic effects of each genetic manipulation in the DC lineages as compared to other cell types, even in the face of eventual indirect effects changing the overall differentiation conditions in the well. Indeed, to rigorously assess how the genetic manipulation under study specifically affected the development of cDC1s or pDCs in a cell-intrinsic manner, we defined a parameter named “differentiation efficacy” that assessed the increase or decrease in the proportion of transduced cells within the cDC1 or pDC populations, normalized to a similar ratio calculated for the whole population of viable cells in the same well (data not shown).

Role of IRF8 in Human cDC1 and pDC Development

We aimed at establishing a proof of concept to validate the suitability of our protocol to assess the impact of the knock-down of candidate genes in human HSCs on their ability to differentiate into cDC1s or pDCs. We used two different shRNAs that we had validated to strongly decrease IRF8 protein expression in THP-1 cells (data not shown). IRF8 inactivation strongly and specifically impaired HSC differentiation into both cDC1s and pDCs (data not shown). The differentiation into CD206⁺ macrophages and CD14 monocytes was not affected (data not shown). In contrast, IRF5 inactivation reduced HSC differentiation into monocytes but not into cDC1s, pDCs and macrophages (data not shown). Conversely to its inactivation, IRF8 overexpression strongly and significantly increased the differentiation of HSCs into cDC1s (data not shown). Thus, these results demonstrate that our culture system recapitulates in vitro the key requirement of IRF8 but not IRF5 for human cDC1 and pDC development. Thus, for the first time to our knowledge, we have achieved combining genetic manipulation of HSCs with an in vitro system recapitulating their physiological differentiation into human cDC1s and pDCs, enabling to identify the underlying molecular mechanisms.

Role of IRFs and Adaptor Molecules in pDC/cDC1 Activation

Next, we harnessed our pipeline to investigate the role of IRF7 versus IRF5 and IRF8 in primary human pDCs derived in vitro from HSCs. We selected two different shRNAs significantly diminishing IRF7 protein expression in transduced pDCs (data not shown). IRF7 inactivation strongly decreased pDC production of IFN-a and IFN-2, but not TNF, in response to stimulation with the Toll-like receptor (TLR)7 ligand R848 (data not shown), whereas this was the case neither for IRF5 (data not shown) nor for IRF8 (data not shown). Thus, primary human pDCs generated in vitro critically require IRF7 for IFN-I/III production in response to viral-type stimuli, whereas IRF5 is dispensable for this function, as observed for primary blood pDCs^{26, 27}, in contrast to the opposite results observed with BDPCN cell lines^{24, 25}. Hence, results obtained with BDPCN cell lines should be considered with caution. Our pipeline will allow checking whether they hold in bona fide primary pDCs derived in vitro from HSCs and amenable to genetic manipulation.

Distinct adaptor molecules promote responses to different TLRs. In mice, MyD88 is required for responses to TLR7, TLR9 and TLR8, including for pDC IFN-I/III production in response to viral-type stimuli^{71, 72}, whereas TRIF is required for TLR3 response, including the unique ability of cDC1s to produce high levels of IFN-III in response to stimulation with polyI:C⁷³. Hence, we used our pipeline to determine which adaptor molecule was required for cytokine production by human pDCs or cDC1s in response to TLR triggering (data not shown). As expected, cDC1 IFN-2 and TNF expression in response to polyI:C stimulation was significantly reduced upon TRIF knock-down (data not shown) , whereas this was not the case for cDC1 TNF production in response to R848 that is known to trigger TLR8 in cDC1s (data not shown). Conversely, in response to R848, cDC1 production of IL-12 and pDC production of IFN-α and IFN-λ were decreased by MYD88 knock-down (data not shown). Thus, these results demonstrate that our culture system recapitulates in vitro the key requirement of TRIF versus MYD88 for TLR3-versus TLR7/8-triggered cytokine responses of human cDC1s and pDCs, and they demonstrate the suitability of our pipeline for gene edition via knock-down in human HSCs in order to study the molecular regulation of the responses of human cDC1s and pDCs to TLR stimulations.

### Role in Human DCs of Transcription Factors

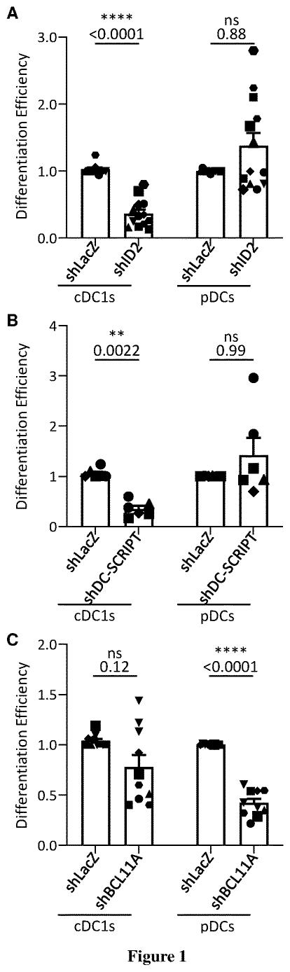

The lack of adequate experimental models has prevented the extension to human DC types of the analysis of the role of transcription factors found to be essential for the differentiation of mouse cDC1s or pDCs. By using our pipeline, we showed here that, both ID2 (FIG. 1A) and DC-SCRIPT (FIG. 1B) promoted human cDC1 development in vitro from HSCs. ID2 knock-down not only strongly decreased cDC1 output in the cultures but conversely led to an increase in pDC output close to significance (FIG. 1A), consistent with the demonstration in mice that ID2 favor cDC1 over pDC differentiation by inhibiting the expression of the master transcription factor instructing pDC fate, TCF4. The cDC1s knocked-down for ID2 harbored a significant but slight decrease in TNF production upon polyI: C stimulation (data not shown), whereas, on the contrary, the cDC1s knocked-down for DC-SCRIPT harbored a significant but slight increase in TNF production upon polyI:C or R848 stimulation (data not shown). Unexpectedly, ID2 knock-down significantly reduced pDC IFN-λ and TNF production in response to R848 (data not shown). BCL11A promoted both pDC development (FIG. 1C) and their production of IFN-α and -λ in response to TLR7 triggering (data not shown). BCL11A knock-down also induced a reduction of cDC1 output in the culture, of lesser intensity than for pDCs but close to significance (data not shown). Thus, these results enabled us to demonstrate critical roles of ID2 and DC-SCRIPT for promoting human cDC1 development, and of BCL11A for promoting human pDC development, for the first time to our knowledge, which is an important translation to humans of previous knowledge discovered by using mutant mouse models.

### Identification of Small GTPases Controlling Human DC Biology

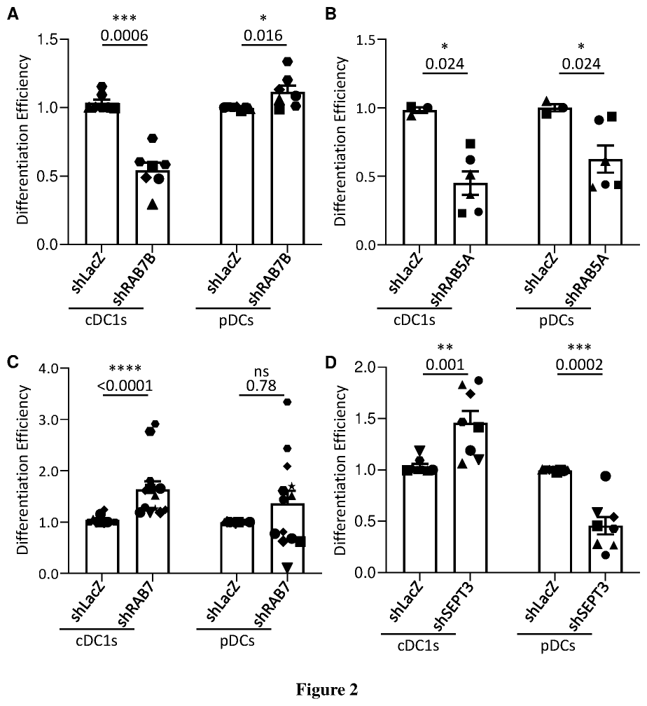

We next screened five small GTPases, RAB7B, RAB15 and SEPT3 because they are selectively expressed at high level in pDCs or cDC1s, but their functions in these cells is largely unknown, as well as RAB5A and RAB7 for comparison. We selected between 1 and 3 shRNA per target, based on their knock-down efficiency as assessed by western blot (data not shown). The knock-down of RAB7B (FIG. 2A) or RAB5A (FIG. 2B) strongly reduced the cDC1 output of the cultures, while slightly increasing or decreasing the pDC output, respectively. The knock-down of RAB7B also slightly increased cDC1 production of TNF while decreasing pDC production of IFN-λ upon R848 stimulation (data not shown). The knock-down of RAB5A also reduced pDC production of IFN-α, IFN-λ and TNF (data not shown). In contrast, RAB7 knock-down (FIG. 1C) promoted the differentiation of HSCs into cDC1s and their production of TNF upon R848 stimulation, without altering the development and activation of pDCs. SEPTIN3 knock-down significantly increased the cDC1 output, and conversely reduced the pDC output; it also decreased pDC production of IFN-α and IFN-λ (FIG. 1D). To our knowledge, this is the first demonstration of the involvement of small GTPases regulating membrane trafficking in the development and activation of human cDC1s or pDCs.

Identification of HIV-1 Restriction Factors in cDC1s/pDCs

MoDCs and cDC2s are resistant to infection by HIV-1 in a large part due to the restriction factor SAMHD1 which deprives the dNTP pool that is required for viral reverse transcription. HIV-1 restriction by SAMHD1 in MoDCs and cDC2s can be overcome by supplementing virus particles with Vpx from HIV-2/SIV, which targets SAMHD1 to proteasome for degradation. In contrast, supplementation by Vpx is insufficient to increase the susceptibility to HIV-1 infection of cDC1s and pDCs, suggesting that these cells express other restriction factors. In cDC1s, RAB15 inhibits one of the earliest phases of the viral life cycle: the fusion of the viral envelope with the cell membrane. RAB15 is also strongly expressed in pDCs, but its function in these cells remains unknown. Another restriction factor protecting cDC1s from HIV-1 infection may be palmitoyl-protein thioesterase 1 (PPT1), since it restricts infection of mouse cDC1s by vesicular stomatitis virus. Thus, we examined whether SAMHD1, RAB15 and PPT1 contributed to the high resistance of cDC1s or pDCs to HIV-1 infection, also testing in parallel some of the genes studied in the previous sections. However, we first had to examine whether SAMHD1, RAB15 or PPT1 modulated the development or activation of human cDC1s or pDCs.

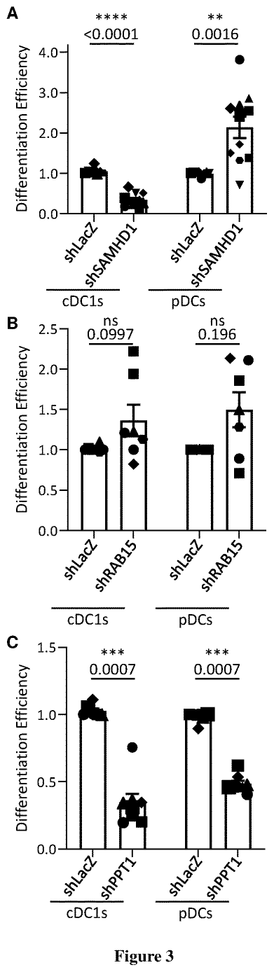

Unexpectedly, SAMHD/knock-down led to a strong and significant decrease in the cDC1 output of the cultures, whereas the pDC output was increased (FIG. 3A). To determine whether this effect could be due to loss of the dNTPase activity of SAMHD1, we tested whether similar results could be obtained by another method also leading to an increase in the cellular dNTP pool: the activation of their synthesis via the salvage pathway upon supplementation of the cultures with dNs. This did not seem to be the case (data not shown). Another mechanism through which SAMHD1 knock-down could increase the pDC output of the cultures is via induction of spontaneous IFN-I responses. Indeed, addition of exogenous IFN-I to human HSC cultures promotes their differentiation into pDCs. This did not seem to be the case since pharmacological blockade of the receptor for IFN-I did not reverse the impact of SAMHD1 knock-down on the cDC1 and pDC output of the cultures (data not shown). Hence, how SAMHD1 controls the differentiation of human HSCs into cDC1s and pDCs remains to be deciphered. SAMHD1 knock-down tended to increase cDC1 cytokine production, reaching significance for TNF in response to R484, whereas pDC cytokine production was unchanged, or decreased in the case of IFN-λ in response to R848 (data not shown). SAMHD1 knock-down slightly increased cDC1 susceptibility to HIV-1 infection, whereas it did not alter pDC permissiveness to the virus (data not shown), consistent with the limited impact of the supplementation of HIV-1 particles with Vpx on their ability to infect cDC1s and pDCs¹¹.

RAB15 knock-down affected neither the development (FIG. 3B) nor the activation (data not shown) of human cDC1s and pDCs. However, it slightly enhanced cDC1 susceptibility to HIV-1 infection, regardless of the presence of Vpx (data not shown), consistent with our previous observations¹¹. RAB15 knock-down increased pDC infection by HIV-1 R5GFP (data not shown). However, unexpectedly, this increase was abolished by the presence of Vpx, implying a negative cross talk between RAB15 and SAMHD1 activities in pDCs. In both cDC1s and pDCs, the increase in HIV-1 infection induced by RAB15 knock-down was abolished upon VSV-G-pseudo-typing of viral particles, showing that the antiviral function of RAB15 was linked to the endocytic route followed by the virus.

PPT1 knock-down strongly and significantly reduced the output of differentiated myeloid cells in our cultures, including cDC1s and pDCs (FIG. 3C), without any observed effect on overall cell expansion. PPT1 knock-down did not affect the activation of cDC1s and pDCs (data not shown). PPT1 knock-down significantly increased the infection by HIV-1 R5GFP of cDC1s regardless of the presence of Vpx, but not of pDCs (data not shown). This increase was abolished upon VSV-G-pseudo-typing of viral particles, showing that the antiviral function of PPT1 in cDC1s was linked to the endocytic route followed by the virus.

Among the other small GTPases that we studied, RAB7 appeared to restrict HIV-1 infection in cDC1s and pDCs (data not shown), in a manner dependent of the virus endocytic entry route since the effects were reduced upon VSV-G-pseudo-typing of viral particles.

Finally, we observed that BCL11A knock-down enhanced HIV-1 infection of cDC1sand pDCs (data not shown), irrespective of the presence of Vpx. Whereas this increase was abrogated in cDC1s upon VSV-G-pseudo-typing of viral particles, this was not the case in pDCs. BCL11A knock-down increased CD4 expression specifically on cDC1s (data not shown).

### Discussion

Here we report a robust and relatively simple workflow to obtain large numbers of gene-silenced human cDC1s and pDCs. By utilizing the pipeline, for the first time to our knowledge, we extended to human cDC1s/pDCs the critical role of the transcription factors ID2 and DC-SCRIPT versus BCL11A in promoting the development of cDC1s versus pDCs. Unexpectedly, we observed that ID2 knock-down impaired pDC production of IFN-α/λ. The underlying mechanisms remain to be investigated.

Three small GTPases of the Rab family, RAB5A, RAB7 and RAB15, are expressed at similar levels between cDC1s and pDCs, but only RAB5A was required for their differentiation. The small GTPases RAB7B and SEPTIN3 are specifically expressed to high levels in cDC1s in both mice and humans, but only the former was required for the optimal development of human cDCIs in vitro from HSCs. The mechanisms by which small GTPases affect the development of cDC1s or pDCs remain to be investigated but may involve altered response to cytokines or growth factors due to deregulation of their receptor expression or signaling, since GTPase control intracellular trafficking including endocytosis/phagocytosis. Unexpectedly, SEPTIN3 knock-down impaired pDC development and their production of IFN-α/λ, despite very low to undetectable expression of the gene in these cells. This suggested that SEPTIN3 might be transiently expressed in a proximal precursor of pDCs in a manner affecting their differentiation and the function of their progeny. More generally, pDC production of IFN-α/λ production seemed more frequently and profoundly impacted by the knock-down of the candidate genes tested than cDC1 cytokine production, suggesting more stringent requirements of various small GTPases for the building in pDCs of the specialized endosomes dedicated to signaling from TLR7/9 triggering to IFN-α/λ production.

Our pipeline not only allowed studying cDC1/pDC ontogeny and cytokine responses in response to TLR triggering; it was also suitable for screening candidate HIV-1 restriction factors and their mechanisms of action in human cDC1s and pDCs that are highly resistant to viral infections¹¹. Here, we showed that BCL11A inhibits HIV-1 infection of pDCs and cDC1s, but probably through distinct mechanisms between these two cell types. Indeed, BCL11A-mediated inhibition of HIV-1 infection was dependent on the route of viral entry specifically in cDC1s, since it was abrogated upon VSVG-pseudo-typing of the virus, and BCL11A knock-down increased specifically on cDC1s the expression of CD4 which is the major entry receptor for HIV-1. Whether the upregulation of CD4 on cDC1s is directly induced by the transcription factor activity of BCL11A remains to be investigated. In contrast, the increase in the infection rate of pDCs upon BCL11A knock-down was not affected by the viral entry route or the presence of Vpx, suggesting an effect at later stages of the viral life cycle. BCL11B, a paralog of BCL11A, can suppress the initial phase of HIV-1 gene transcription in human microglial cells or of HIV-1 long terminal repeats in T cells. Whether BCL11A can regulate the transcription of HIV-1 proviruses in pDCs remains to be investigated. Using our pipeline will also help understand how other viruses representing a threat for human health interact with human DC types.

Our work focused on using shRNA for target gene knock-down. Further applications of our pipeline could be achieving gene knock-down in primary human cDC1s/pDCs, by combining lentivector-based delivery of guide RNA with electroporation-based delivery of Cas mRNA, as was previously achieved in other experimental systems of in vitro differentiation of human immune cells from HSCs.

In conclusion, we were successful in setting-up a robust pipeline to genetically edit human cDC1s and pDCs, enabling to test the impact of the knock-down of candidate genes on their biology. This allowed us identifying novel mechanisms controlling human cDC1/pDC development, cytokine production or susceptibility to HIV-1 infection. This technological breakthrough should allow conducting larger scale genetic screens to advance our knowledge on the functions of human cDC1s/pDCs and their molecular regulation, with the perspective to manipulate them for designing complementary approaches to boost existing vaccination or immunotherapies against viral infections or cancer.

### References

Throughout this application, various references describe the state of the art to which this invention pertains. The disclosures of these references are hereby incorporated by reference into the present disclosure.
1. Vu Manh, T. P., Bertho, N., Hosmalin, A., Schwartz-Cornil, I. & Dalod, M. Investigating Evolutionary Conservation of Dendritic Cell Subset Identity and Functions. Front Immunol 6, 260 (2015).
2. Guilliams, M. et al. Dendritic cells, monocytes and macrophages: a unified nomenclature based on ontogeny. Nat Rev Immunol 14, 571-578 (2014).
3. Dalod, M. & Scheu, S. Dendritic cell functions in vivo: A user's guide to current and next-generation mutant mouse models. Eur J Immunol 52, 1712-1749 (2022).
4. Crozat, K. et al. Comparative genomics as a tool to reveal functional equivalences between human and mouse dendritic cell subsets. Immunol Rev 234, 177-198 (2010).
5. Robbins, S. H. et al. Novel insights into the relationships between dendritic cell subsets in human and mouse revealed by genome-wide expression profiling. Genome Biol 9, R17 (2008).
6. Guo, Y. et al. Herpes simplex virus encephalitis in a patient with complete TLR3 deficiency: TLR3 is otherwise redundant in protective immunity. J Exp Med 208, 2083-2098 (2011).
7. Philippot, Q. et al. Human IL-23 is essential for IFN-gamma-dependent immunity to mycobacteria. Sci Immunol 8, eabq5204 (2023).
8. Rosain, J. et al. Human IRF1 governs macrophagic IFN-gamma immunity to mycobacteria. Cell 186, 621-645 e633 (2023).
9Alcantara-Hernandez, M. et al. High-Dimensional Phenotypic Mapping of Human Dendritic Cells Reveals Interindividual Variation and Tissue Specialization. Immunity 47, 1037-1050 e1036 (2017).
10. Balan, S. et al. Human XCR1+ dendritic cells derived in vitro from CD34+ progenitors closely resemble blood dendritic cells, including their adjuvant responsiveness, contrary to monocyte-derived dendritic cells. J Immunol 193, 1622-1635 (2014).
11. Silvin, A. et al. Constitutive resistance to viral infection in human CD141(+) dendritic cells. Sci Immunol 2 (2017).
12. Adema, G. J., de Vries, I. J., Punt, C. J. & Figdor, C. G. Migration of dendritic cell based cancer vaccines: in vivo veritas? Curr Opin Immunol 17, 170-174 (2005).
13. Plantinga, M. et al. Conventional and monocyte-derived CD11b(+) dendritic cells initiate and maintain T helper 2 cell-mediated immunity to house dust mite allergen. Immunity 38, 322-335 (2013).
14. Zhou, Y. et al. Vaccine efficacy against primary and metastatic cancer with in vitro-generated CD103(+) conventional dendritic cells. J Immunother Cancer 8 (2020).
15. Sittig, S. P., de Vries, I. J. M. & Schreibelt, G. Primary Human Blood Dendritic Cells for Cancer Immunotherapy-Tailoring the Immune Response by Dendritic Cell Maturation. Biomedicines 3, 282-303 (2015).
16. Westdorp, H. et al. Blood-derived dendritic cell vaccinations induce immune responses that correlate with clinical outcome in patients with chemo-naive castration-resistant prostate cancer. J Immunother Cancer 7, 302 (2019).
17. Wimmers, F., Schreibelt, G., Skold, A. E., Figdor, C. G. & De Vries, I. J. Paradigm Shift in Dendritic Cell-Based Immunotherapy: From in vitro Generated Monocyte-Derived DCs to Naturally Circulating DC Subsets. Front Immunol 5, 165 (2014).
18. Bigley, V., Cytlak, U. & Collin, M. Human dendritic cell immunodeficiencies. Semin Cell Dev Biol 86, 50-61 (2019).
19. Luo, X. L. & Dalod, M. The quest for faithful in vitro models of human dendritic cells types. Mol Immunol 123, 40-59 (2020).
20. Balan, S. et al. Large-Scale Human Dendritic Cell Differentiation Revealing Notch-Dependent Lineage Bifurcation and Heterogeneity. Cell Rep 24, 1902-1915 e1906 (2018).
21. Luo X, Balan S, Arnold-Schrauf C, Dalod M. In Vitro Generation of Human Cross-Presenting Type 1 Conventional Dendritic Cells (cDC1s) and Plasmacytoid Dendritic Cells (pDCs). Methods Mol Biol. 2023; 2618:133-145.

1. An in vitro or ex vivo method for inducing the differentiation of human hematopoietic stem cells (HSCs) into human type 1 conventional dendritic cells (cDC1s) comprising:
i) transfecting or transducing a population of human hematopoietic stem cells (HSCs) with at least one gene selected from the group consisting in IRF8, ID2, DC-SCRIPT, RAB7B, RAB5A, PPT1 and SAMHD1 and/or with an inhibitor of the expression of a gene selected in the group consisting from RAB7 and SEPT3; and
ii) culturing the population of HSCs obtained at step i) in at least one dendritic cell differentiation medium to produce a population of human dendritic cells enriched in type 1 conventional dendritic cells (cDC1s).

2. The method of claim 1, wherein the population of hematopoietic stem cells (HSCs) is transfected of transduced with IRF8, ID2 and/or DC-SCRIPT.

3. An in vitro or ex vivo method for inducing the differentiation of human hematopoietic stem cells (HSCs) into human plasmacytoid dendritic cells (pDCs) comprising:
i) transfecting or transducing a population of human hematopoietic stem cells (HSCs) in vitro or ex vivo with at least one gene selected from IRF8, BCL11A, PPT1 and SEPT3, and/or with an inhibitor of the expression of a gene selected in the group consisting from ID2 and SAMHD1; and
ii) culturing the population of HSCs obtained at step i) in at least one dendritic cell differentiation medium to produce a population of human dendritic cells enriched with plasmacytoid dendritic cells (pDCs).

4. The method of claim 3, wherein the population of hematopoietic stem cells (HSCs) is transfected of transduced with IRF8, BCL11A and/or PPT1.

5. An in vitro or ex vivo method for increasing the immune activity of human type 1 conventional dendritic cells (cDC1s) comprising:
i) transfecting or transducing a population of human hematopoietic stem cells (HSCs) in vitro or ex vivo with at least one gene selected from the group consisting in SAMHD1, TRIF, MYD88, DC-SCRIPT, CD40, IL12B and SEPT3 and/or an inhibitor of the expression of at least one gene selected from the group consisting in BCL11A, RAB7B, RAB7, PD-L1, PD-L2, TRIM3 or IL10; and
ii) culturing the population of HSCs obtained at step i) in at least one dendritic cell differentiation medium to produce a population of human dendritic cells with more immunogenic type 1 conventional dendritic cells (cDC1s).

6. An in vitro or ex vivo method for increasing the immune activity of human plasmacytoid dendritic cells (pDCs) comprising: the steps of
i) transfecting or transducing a population of human hematopoietic stem cells (HSCs) in vitro or ex vivo with at least one gene selected from the group consisting in IRF7, MYD88, BCL11A, RAB7, SEPT3, CD40 and IL12B and/or an inhibitor of the expression of at least one gene selected from the group consisting in RAB5A, PD-L1, PD-L2, ICOS-L and IL-10; and
ii) culturing the population of HSCs obtained at step i) in at least one dendritic cell differentiation medium to produce a population of human dendritic cells with more immunogenic plasmacytoid dendritic cells (pDCs).

7. The method of claim 1, wherein the hematopoietic stem cells is human cord blood hematopoietic stem cells.

8. The method of claim 1, wherein the gene and/or the inhibitor of expression is transfected or transduced into HSC via at least one vector, and especially via a viral vector and in particularly a lentivirus vector.

9. The method of claim 1, wherein the dendritic cell differentiation medium comprises Fms-related tyrosine kinase 3 ligand (FTL3L), stem cell factor (SCF) and thrombopoietin (TPO) and furthermore an OP9/OP9-DL1 feeder layer.

10. A population of human dendritic cells enriched in type 1 conventional dendritic cells (cDC1s) produced by a method of claim 1.

11. A therapeutic method comprising providing a subject with the population of human dendritic cells enriched in type 1 conventional dendritic cells (cDC1s) according to claim 10.

12. The therapeutic method according to claim 11, wherein the therapy is adoptive cell therapy in a subject in need thereof.

13. A method of treating infectious disease in a subject in need thereof, said method comprising administering to the subject a therapeutically effective amount of the population of human dendritic cells enriched in type 1 conventional dendritic cells (cDC1s) according to claim 10.

14. The method according to claim 13, wherein the infectious disease is caused by an intracellular pathogen.

15. A method of treating cancer in a subject in need thereof, said method comprising administering to the subject a therapeutically effective amount of the population of human dendritic cells enriched in type 1 conventional dendritic cells (cDC1s) according to claim 10.

16. A population of human dendritic cells enriched in plasmacytoid conventional dendritic cells (pDCs) produced by a method of claim 3.

17. A therapeutic method comprising providing a subject with the population of human dendritic cells enriched in plasmacytoid conventional dendritic cells (pDCs) according to claim 16.

18. The therapeutic method according to claim 17, wherein the therapy is adoptive cell therapy in a subject in need thereof.

19. A method of treating infectious disease in a subject in need thereof, said method comprising administering to the subject a therapeutically effective amount the population of human dendritic cells enriched in plasmacytoid conventional dendritic cells (pDCs) according to claim 16.

20. A method of treating cancer in a subject in need thereof, said method comprising administering to the subject a therapeutically effective amount of the plasmacytoid dendritic cells according to claim 16.
